(19) United States
(12) Patent Application Publication
(10) Pub. No.: US 2020/0265950 A1
(43) Pub. Date: Aug. 20, 2020

(54) BIOLOGICAL INFORMATION PROCESSING SYSTEM, BIOLOGICAL INFORMATION PROCESSING METHOD, AND COMPUTER PROGRAM RECORDING MEDIUM

(71) Applicant: NEC CORPORATION, Tokyo (JP)

(72) Inventors: Toshinori HOSOI, Tokyo (JP); Yuji OHNO, Tokyo (JP); Masahiro KUBO, Tokyo (JP); Masahiro HAYASHITANI, Tokyo (JP); Shigemi KITAHARA, Tokyo (JP)

(73) Assignee: NEC CORPORATION, Tokyo (JP)

(21) Appl. No.: 16/643,363

(22) PCT Filed: Aug. 22, 2018
(86) PCT No.: PCT/JP2018/030970

§ 371 Date: Feb. 28, 2020

(30) Foreign Application Priority Data
Aug. 30, 2017 (JP) 2017-165605

(51) Int. Cl. G16H 40/63; G06N 20/00; A61B 5/16; A61B 5/00; A61B 5/0205; A61B 5/0476; A61B 5/0456

(52) U.S. Cl. CPC G16H 40/63; G06N 20/00; A61B 5/165; A61B 5/021; A61B 5/02055; A61B 5/0476; A61B 5/0456; A61B 5/7264

(57) ABSTRACT

The present invention can predict the occurrence of target-patient problem behavior prior the occurrence of such problem behavior. A biological information processing system includes: a feature calculation unit that calculates, from input biological information of the target patient, detection-use feature time-series data which indicates a feature related to the target patient: and an agitation detection unit that processes the detection-use feature time-series data on the basis of a pre-acquired discrimination parameter, that calculates the current agitation score of the target patient, and that detects the current agitation state of the target patient prior to the target-patient problem behavior.

### TECHNICAL FIELD

The present invention relates to a biological information processing system, a biological information processing method, and a computer program recording medium.

### BACKGROUND ART

Patent Literature 1 discloses a user monitoring system which predicts the onset of an adverse condition of a user. More specifically, Patent Literature 1 discloses the user monitoring system which comprises a first sensor, a second sensor, and a controller. The first sensor is provided to a user support apparatus such as a bed and detects first information corresponding to a feature of the user support apparatus. The second sensor detects second information corresponding to a physiological characteristic of the user. The controller calculates an indicator based on the first information and the second information according to a user's adverse condition prediction algorithm. The controller is configured to alert nurses or caregivers (which will hereinafter be called nursing/caregiving workers) when the indicator exceeds a threshold.

Patent Literature 1 also discloses to detect, by the second sensor, a heartbeat, body temperature, and so on and to decide whether the user is alert, responsive to voice, responsive to pain, or unresponsive.

However, Patent Literature 1 does not specifically describe the user's adverse condition prediction algorithm.

Patent Literature 2 discloses a dementia risk determination system configured to generate sleep data including the depth of sleep and body motion change based on biological data of a subject during sleeping and to determine dementia risks by comparison with an existing pattern.

Furthermore, Patent Literature 3 discloses a system which comprises a first motion sensor, a second motion sensor, and a pattern analysis module. The first motion sensor detects motions of a subject on a bed. The second motion sensor is arranged in a second object for the subject to rest. The pattern analysis module receives data from the first and the second sensors to monitor clinical symptoms.

Patent Literature 4 discloses a monitoring system for monitoring a patient and detecting delirium of the patient. Specifically, Patent Document 4 discloses an evaluation unit for detecting motion events of the patient from image data of the patient and for classifying the detected motion events into delirium-typical motion events and non-delirium-typical motion events. Furthermore, Patent Literature 4 clarifies a delirium determination unit for determining, from the duration of delirium-typical motion events and so on evaluated by the evaluation unit, a delirium score indicating the likelihood and/or intensity of delirium of the patient. Herein, the “delirium” is one of disturbance of consciousness and is a state where an unusual behavior, speech, and excitement are observed due to a temporary sense of unease.

### CITATION LIST

### Patent Literatures

PL 1: JP 2011-120874 A

PL 2: JP 2016-22310 A

PL 3: JP 2013-154190 A

PL 4: JP 2014-528514 A

### SUMMARY OF INVENTION

### Technical Problem

Patent Literature 1 merely discloses to monitor a condition of the patient being the user him/herself. This applies to Patent Literatures 2 and 3 also.

In addition, the monitoring system of Patent Literature 4 judges that a patient is a delirious patient when the patient shows a pronounced hyperactive behavior, a pronounced hypoactive behavior and/or significantly often delirium-typical movements. Furthermore, Patent Literature 4 describes the monitoring system for determining a delirious condition from the image of the patient. Specifically, Patent Literature 4 describes that the monitoring system compares the delirium score of the patient (delirious patient) in the delirious condition with a particular threshold determined with reference to a score of a non-delirious patient and generates an alarm when the delirium score exceeds the threshold. However, Patent Literature 4 never describes any problem behavior of the delirium patient after the delirium is detected.

Furthermore, in a case where the delirium score is compared with the particular threshold as in the monitoring system of Patent Document 4, there is a disadvantage that a detection rate of the problem behavior after detection of the delirium is low and a lot of errors occur because occurrence of delirium itself differs among individual patients.

As will be understood from the above, any of Patent Literatures 1 to 4 never takes account of increase of a burden and a workload imposed on the nursing/caregiving workers by the problem behavior of the patient. That is, Patent Literatures 1 to 4 do not take account of the problem behavior of the patient towards the nursing/caregiving workers at all.

In this description, the “problem behavior” is, for example, a behavior of the patient that imposes a burden on the nursing/caregiving workers. Specifically, the problem behavior is a behavior of the patient that imposes a burden and a trouble on nursing/caregiving services. More specifically, the problem behavior is a behavior of the patient such that the patient sits up on the bed, removes a fence of the bed, leaves the bed, walks by oneself, wanders around, goes to another floor in a hospital, falls down from the bed, touches and evulses a drip, a tube or the like, utters a strange sound, verbally abuses, or uses violence.

On the other hand, in order to deal with the problem behavior of the patient, the nursing/caregiving workers for the patient may spare as much time as 20 to 30 percent of working hours. For example, the problem behavior of the patient such as falling down from the bed, evulsion of the tube, utterance of the strange sound, an action of violence, or a bed-leaving behavior is a behavior with a lot of risks not only for the patient him/herself but also for the nursing/caregiving workers. In a state where the nursing/caregiving workers spare a lot of time for such problem behavior of the patient, this results in compression of a time for the nursing/caregiving workers to concentrate on care services as their primary duty.

Even if the problem behavior is detected after occurrence thereof, it is impossible to suppress the occurrence of the problem behavior and this may lead to an accident such as an injury of the patient or the nursing/caregiving workers.

Actually, for the patient who has exhibited the problem behavior, treatment of administering a strong sedative drug or treatment of restraining a body by a restraint instrument or the like is taken. In a case of taking such an after-treatment, a rapid practice of rehabilitation is inhibited, recovery is significantly delayed, and prognosis often becomes worse.

Thus, the above-mentioned Patent Literatures 1 to 4 have a problem that the patient exhibiting the problem behavior cannot be predicted prior to occurrence of the problem behavior in question.

It is an object of the present invention to resolve the above-mentioned problem and to provide a biological information processing system/processing method capable of predicting, before occurrence of a problem behavior of a patient, occurrence of the problem behavior in question.

It is another object of the present invention to provide a computer program recording medium which can be used in the biological information processing system.

### Solution to Problem

According to a first aspect of the present invention, there is provided a biological information processing system comprising a feature calculation unit configured to calculate, from input biological information of a target patient, detection-use feature time-series data indicative of a feature related to the target patient; and an agitation detection unit configured to process the detection-use feature time-series data on the basis of a discrimination parameter which is preliminarily acquired, to calculate a current agitation score of the target patient, and to detect a current agitation state of the target patient prior to a problem behavior of the target patient.

According to a second aspect of the present invention, there is provided a biological information processing system comprising a feature calculation unit configured to calculate, from input biological information of a target patient, detection-use feature time-series data indicative of a feature related to the target patient; a discrimination parameter storage unit configured to store a discrimination parameter which is preliminarily acquired; and a discrimination parameter renewal unit configured to process the detection-use feature time-series data on the basis of the discrimination parameter to renew the discrimination parameter.

According to a third aspect of the present invention, there is provided a biological information processing method comprising calculating, from input biological information of a target patient, detection-use feature time-series data indicative of a feature related to the target patient; and processing the detection-use feature time-series data on the basis of a discrimination parameter which is preliminarily acquired, calculating a current agitation score of the target patient, and detecting a current agitation state of the target patient prior to a problem behavior of the target patient.

According to a fourth aspect of the present invention, there is provided a recording medium recording a computer program which causes a computer to execute the steps of calculating, from input biological information of a target patient, detection-use feature time-series data indicative of a feature related to the target patient; and processing the detection-use feature time-series data on the basis of a discrimination parameter which is preliminarily acquired, calculating a current agitation score of the target patient, and detecting a current agitation state of the target patient prior to a problem behavior of the target patient.

### Advantageous Effect of the Invention

According to the present invention, it is possible to greatly reduce a workload and a burden imposed on nursing/caregiving workers who deal with a problem behavior of a patient because the problem behavior is predicted prior to the occurrence thereof.

### BRIEF DESCRIPTION OF THE DRAWINGS

FIG. 1 is a block diagram for describing a principle of the present invention;

FIG. 2 is a flow chart for describing an operation in FIG. 1;

FIG. 3 is a block diagram for illustrating a biological information processing system according to a first example embodiment of the present invention;

FIG. 4 is a block diagram for illustrating a system which is used in preparation of a learned discrimination parameter storage unit illustrated in FIG. 3;

FIG. 5 is a block diagram for illustrating a biological information processing system according to a second example embodiment of the present invention;

FIG. 6 is a block diagram for illustrating an example in which the system illustrated in FIG. 4 is applied to other biological information; and

FIG. 7 is a block diagram for illustrating an example of hardware configuration of the biological information processing systems according to the first and the second example embodiments of the present invention.

### DESCRIPTION OF EMBODIMENTS

According to observation of the present inventors, it has been confirmed that, at least in a case of a patient related to neurosurgery, the patient is often turned into an agitation state (agitation) where his/her behavior is excessive and restless, prior to actually exhibiting any problem behavior. Herein, the “agitation” includes not only the state where the behavior is excessive and restless but also a state where the patient is not calm and a state where the patient cannot normally control his/her mind. In addition, the term “agitation” in this description includes delirium because the agitation occurs due to physical pain, the delirium, and uneasiness.

Specific behaviors of the patient in the agitation are behaviors such as continuously moving hands and feet without any reason, shaking his/her body, unnaturally concentrating to some action, making a logically indistinct statement, not listening to the nursing/caregiving workers, and so on. Furthermore, in this description, a behavior which is not harmful to the patient, for example, which follows a patient's desire to urinate is included in the agitation.

In any event, it is supposed that, if the agitation can be automatically detected before the occurrence of the problem behavior of the patient, for example, about ten minutes before and notified to the nursing/caregiving workers, the burden on the nursing/caregiving workers can be remarkably reduced.

The present invention is based on the above-mentioned knowledge. Specifically, the present invention resides in detecting an agitation state of the patient to detect and predict the problem behavior before the occurrence thereof by calculating, using a machine learning technique, similarity and dissimilarity between a temporal change of biological information of the patient and a temporal change pattern as a predictor of a problem having occurred in the past.

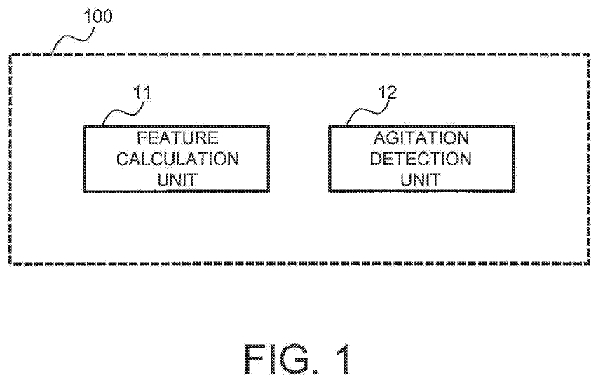

Referring now to FIG. 1, a biological information processing system 100 according to the present invention will be described specifically. The illustrated biological information processing system 100 is supplied from sensors (not shown) or the like with, as an input signal, biological information of a patient to be targeted (target patient) that is obtained by sensing. The biological information processing system 100 comprises a feature calculation unit 11 for calculating detection-use time-series data for use in detection processing, indicative of a current feature of the biological information and an agitation detection unit 12 including a model (discrimination parameter) which is obtained from a relationship between a plurality of pieces of past biological information and a past agitation/non-agitation state. Therefore, the agitation detection unit 12 comprises a storage unit for storing the past discrimination parameter preliminarily acquired, as will later be described. Herein, the past biological information may be biological information of the target patient or may be biological information of patients other than the target patient.

When the agitation detection unit 12 illustrated in FIG. 1 receives, from the feature calculation unit 11, the detection-use time-series data calculated on the basis of the biological information, the agitation detection unit generates, as a current agitation score, a discrimination result indicative of a current agitation/non-agitation state of the patient using the discrimination parameter and notifies the nursing/caregiving workers or the like. That is, the agitation detection unit 12 automatically detects the current agitation/non-agitation state of the patient as the discrimination result using the discrimination parameter and the detection-use time-series data and notifies the nursing/caregiving workers or the like. Therefore, the agitation detection unit 12 may be called a notification unit for detecting the current agitation/non-agitation state of the target patient to notify the nursing/caregiving workers.

Detection of the agitation/non-agitation state of each patient makes it possible to preliminarily predict the occurrence of the problem behavior of each patient that may possibly occur during agitation. Accordingly, the biological information processing system 100 according to the present invention may be called a biological information detection and prediction system for processing the biological information to predict the problem behavior of the patient.

The illustrated agitation detection unit 12 includes the discrimination parameter generated and prepared in a learning phase of the machine learning. Furthermore, the agitation detection unit 12 carries out operation of discriminating or regressing the detection-use time-series data per target patient as the input signal into two classes of agitation/non-agitation using the learned discrimination parameter.

More specifically, the feature calculation unit 11 illustrated in FIG. 1 receives the current biological information of the target patient and calculates the detection-use feature time-series data X(t) (time-series data X(t)) indicative of the feature of the biological information of the target patient in question to produce the data. Herein, it is assumed that information relating to a heartbeat is used as the biological information. The heartbeat means a beat of the heart and will be described as an equivalent of pulsation in the present description.

In addition, a heart rate represents the number of beats of the heart in a given time interval (e.g. one-minute interval). In a medical scene, the heart rate is often calculated using an electrocardiogram including a plurality of waveforms such as a P wave, a Q wave, an R wave, an S wave, and a T wave. For example, the heart rate is calculated by dividing a predetermined number (e.g. 300 or 1500) by an interval of the R wave (RR interval) represented in the electrocardiogram.

On the other hand, if the RR interval is measured, the heart rate in the one-minute interval can be calculated by multiplying a reciprocal of the RR interval by 60. It is therefore possible to calculate the heart rate also in a case where the reciprocal (RR interval) of a heartbeat value is given as the input signal which is the biological information. In addition, if the heart rate in a predetermined time interval is measured, it is also possible to use the heart rate in question as the input signal.

Taking the above into account, it is assumed that the feature calculation unit 11 of the biological information processing system 100 according to the present invention is supplied from a heartbeat sensor or the like with the information relating to the heartbeat, the heartbeat value and/or the reciprocal of the heartbeat value as the biological information. The biological information supplied to the feature calculation unit 11 may be analog information or may be digital information.

Herein, it is assumed that the input signal being the current biological information of the target patient is supplied from the heartbeat sensor or the like to the feature calculation unit 11 in the form of the time-series data, that is, a digital signal. Accordingly, the input signal being the current biological information is given from the heartbeat sensor to the feature calculation unit 11 as the digital information represented by the time-series data.

More specifically, when the heartbeat value at a time instant t (t=1, 2, . . . T) is represented by h_t, time-series data of the heartbeat value h_t or time-series data of the reciprocal r_t thereof (RR interval) is supplied to the feature calculation unit 11 as the time-series data X(t) of the biological information. In this event, the feature calculation unit 11 may be supplied with only the time-series data of the heartbeat value h_t or only the time-series data of the reciprocal r_t (RR interval) of the heartbeat value. In addition, the feature calculation unit 11 may be supplied with both of the time-series data.

Hereinafter, as one example, it is assumed that an output portion of the heartbeat sensor as a sensor, which obtains the reciprocal (RR interval) of the heartbeat value is connected to the biological information processing system 100 via wire or wireless connection which is not shown. In this event, description will mainly be made about a case where the time-series data of the reciprocal r_t of the heartbeat value is supplied from the heartbeat sensor to the biological information processing system 100.

The illustrated feature calculation unit 11 comprises a plurality of filters having bands which are different from one another (e.g. a total of 500 types of filters, such as a plurality of bandpass filters having different passhands, a differential filter, and so on). The feature calculation unit 11 carries out, by using the plurality of filters, leveling processing or differential processing on the input biological information, combines a plurality of values obtained, and produces detection-use time-series data Y(t) representing the feature of the heartbeat being the biological information. The detection-use time-series data Y(t) representing a filtered feature is a feature vector. Hereinafter, the time-series data related to past biological information is marked with “′” in order to distinguish between the time-series data related to the current biological information from the target patient and the time-series data related to the past biological information.

On the other hand, the agitation detection unit 12 is provided with the discrimination parameter which is obtained by machine-learning past time-series data Y′(t) indicative of the feature obtained from time-series data X′(t) of the past biological information and a lot of data of the past agitation/non-agitation state of the patient. That is, the discrimination parameter is generated by machine-learning a first past feature (first time-series data Y′(t) for learning processing) obtained from the past biological information (first past time-series data X′(t)) sensed in the agitation state and a second past feature (second time-series data Y′(t) for learning processing) obtained from the past biological information (second past time-series data X′(t)) sensed in the non-agitation state. That is, the detection-use time-series data Y(t) indicative of the feature is multiplied by the preliminarily generated discrimination parameter and, as a result, the agitation detection unit 12 produces the agitation score (discriminated result) determined by the discrimination parameter and the detection-use time-series data Y(t) and indicative of the current agitation/non-agitation state.

As described above, the biological information processing system 100 detects and notifies the current agitation/non-agitation state of the target patient in accordance with the discrimination parameter which is obtained by learning the current biological information time-series data X(t) per target patient and the past biological information time-series data X′(t). Therefore, the biological information processing system 100 can detect the agitation state of each patient individually and can notify the nursing/caregiving workers of the possibility of occurrence of the problem behavior prior to the occurrence of the problem behavior of the patient. In addition, since the machine learning technique is used, the biological information processing system 100 according to the present invention has a higher detection rate with an increase in accumulated data and a progress of learning and has an advantage that it is possible to greatly reduce a burden imposed on the nursing/caregiving workers dealing with the problem behavior of the patient.

For convenience of explanation, the feature calculation unit 11 and the agitation detection unit 12 are separately described in FIG. 1. However, the feature calculation unit 11 and the agitation detection unit 12 may be configured by a plurality of processors individually carrying out the above-mentioned processing or may be configured by a single processor which operates by a computer program carrying out the above-mentioned processing.

Herein, the “biological information” is information related to a living body and obtained by sensors or the like. In addition, the “biological information” is, for example, data (vital signs) obtained by biosensing. Specifically, the “biological information” includes at least one piece of biological information such as a heartbeat (pulsation), breathing, blood pressure, deep-body temperature, a level of consciousness, skin temperature, skin conductance response (Galvanic Skin Response (GSR)), a skin potential, a myoelectric potential, an electrocardiographic waveform, an electroencephalographic waveform, a sweating amount, a blood oxygen saturation level, a pulse waveform, optical brain function mapping (Near-Infrared Spectroscopy (NIRS)), a urine volume, and pupil reflex, but is not limited thereto.

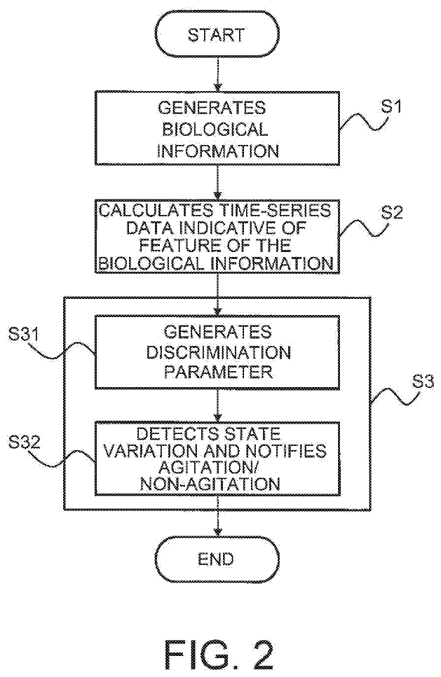

By using a flowchart illustrated in FIG. 2, operation of the biological information processing system 100 according to FIG. 1 will be described. The current biological information relating to the patient is supplied from the sensor to the feature calculation unit 11 illustrated in FIG. 1 (step S1). The feature calculation unit 11 calculates the detection-use feature time-series data Y(t) on the basis of the current biological information of the patient that is received from the sensor (step S2). In this example, the feature calculation unit 11 calculates, on the basis of the current biological information of the patient, the detection-use feature time-series data Y(t) and supplies the data to the agitation detection unit 12.

The agitation detection unit 12 calculates a current agitation score on the basis of the detection-use feature time-series data Y(t) and a preliminarily acquired discrimination parameter, detects a current agitation/non-agitation state of the patient, and notifies the nursing/caregiving workers (step S3).

In the example being illustrated, the storage unit of the agitation detection unit 12 stores the discrimination parameter preliminarily obtained by machine learning. The discrimination parameter is preliminarily generated on the basis of the time-series data Y′(t) for learning processing, indicative of the past feature obtained from the past biological information time-series data X′(t) and the past agitation/non-agitation state data. The agitation detection unit 12 calculates the current agitation score on the basis of the supplied detection-use feature time-series data Y(t) and the discrimination parameter (step S31). Furthermore, the agitation detection unit 12 notifies the nursing/caregiving workers of the current agitation score (step S32). The current agitation score indicates a current agitation/non-agitation state of the patient as the target patient.

In the example illustrated in FIGS. 1 and 2, the biological information processing system 100 can detect the current agitation/non-agitation state of the patient by the current agitation score before the target patient exhibits the problem behavior actually. As described above, it is possible to detect that the patient in question is in the agitation state before the occurrence of the problem behavior of the patient. Therefore, the nursing and caregiving workers can predict the problem behavior of the patient and plans a countermeasure for the problem behavior of the patient. For example, prior to the occurrence of the problem behavior, the nursing/caregiving workers can make preparations which would be required in a case where the problem behavior occurs. Furthermore, it is also possible to predict the occurrence of the problem behavior per patient and to preliminarily prepare a measure suitable for each patient. Accordingly, it is possible to greatly reduce a burden on the nursing/caregiving workers. In addition, if it is possible to control the problem behavior prior to the occurrence thereof, a large effect is obtained in rehabilitation of the patient him/herself.

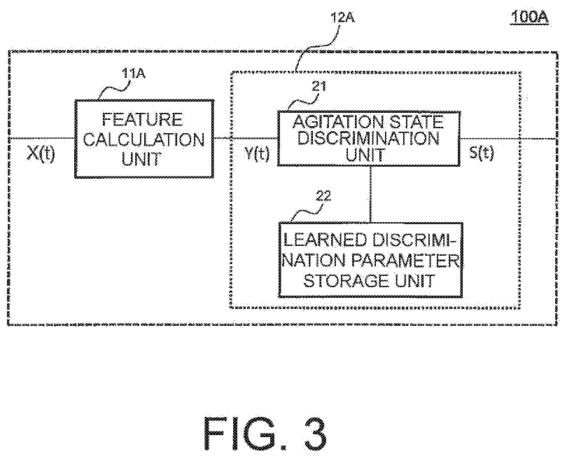

Referring to FIG. 3, description will proceed to a biological information processing system 100A according to a first example embodiment of the present invention. The biological information processing system 100A illustrated in FIG. 3 comprises a feature calculation unit 11A which corresponds to the calculation unit 11 illustrated in FIG. 1. It is assumed that the feature calculation unit 11A is supplied with, as the current biological information of the target patient, the current time-series data X(t) indicative of a heartbeat of the patient and obtained by sensing of the sensor such as an electrocardiograph.

The input time-series data X(t) may be a heartbeat value h(t) at a time instant (t=1, 2 . . . T) and/or time-series data (r_t(=C/h_t): C being a constant) of a heartbeat interval which is a reciprocal of the heartbeat value. Herein, it is assumed that, as the current time-series data X(t), the time-series data (r_t) is given from the heartbeat sensor or the like.

The feature calculation unit 11A calculates a difference between the time-series data r_t at different time instants t1 and t2. Hereinafter, the feature calculation unit 11A successively calculates differences between time-series data r_t at time instants t2, . . . which are different from one another, and produces a train of the differences obtained by calculation as the detection-use feature time-series data Y(t).

Furthermore, the feature calculation unit 11A calculates, in addition to the differences of the time-series data r_t (a train of numerical values) within various predetermined time intervals, difference values (a train of numerical values) of the time-series data r_t which are calculated in various time intervals, and calculates a group of numerical values obtained by combining a minimum value min of these difference values and a maximum value max of these difference values. The group of numerical values is supplied from the feature calculation unit 11A to an agitation detection unit 12A as the detection-use feature time-series data Y(t) indicative of the heartbeat, i.e., the feature vector.

The feature vector Y(t) being the detection-use feature time-series data may include a ratio of heartbeat interval data r_t at the different time instants t1 and t2 or the heartbeat interval data r_1 at the time instant t1. Thus, it is needless to say that the feature vector calculated by the feature calculation unit 11A is not limited to the above-mentioned time-series data. Furthermore, as already described, the above-mentioned detection-use feature time-series data Y(t) may be obtained by filtering the current time-series data X(t) using a plurality of filters (bandpass filters, differential filter, and so on) and then carrying out calculation.

The illustrated agitation detection unit 12A comprises an agitation state discrimination unit 21 and a learned discrimination parameter storage unit 22. The learned discrimination parameter storage unit 22 stores a learned discrimination parameter (discrimination parameter) which is calculated in a learning phase of the machine teaming. Herein, the learned discrimination parameter storage unit 22 stores the learned discrimination parameter generated on the basis of the feature time-series data Y′(t) for learning processing, obtained by calculating from the past biological information time-series data X′(t) and data indicative of a past agitation/non-agitation state. The learned discrimination parameter storage unit 22 may be provided inside the agitation detection unit 12A together with the agitation state discrimination unit 21 as shown in FIG. 3 or may be externally connected to the agitation detection unit 12A. That is, the learned discrimination parameter storage unit 22 storing the discrimination parameter may be marketed as a single unit.

The agitation state discrimination unit 21 calculates and produces a current agitation score S(t) of the target patient on the basis of the detection-use feature time-series data Y(t) received from the feature calculation unit 11A and the learned discrimination parameter read out from the learned discrimination parameter storage unit 22. Inasmuch as the current agitation score produced by the agitation state discrimination unit 21 represents the current agitation/non-agitation state of the target patient, the agitation state discrimination unit 21 carries out operation of discriminating the current agitation state of the target patient.

Furthermore, the agitation score S(t) indicative of the current agitation/non-agitation may be produced in the form of a binary signal (1/0) indicative of one of the agitation/non-agitation or may be produced in the form of a score having a numerical value in a range between 0 and 1, both inclusive, that represents the degree of similarity therebetween. In this event, the numerical value of the score represents agitation as the value is closer to 1 and non-agitation as the value is closer to 0. It is noted that the manner how to produce the current agitation score S(t) is not limited to the above and may be any manner as far as agitation or near-agitation, and non-agitation or near-non-agitation are expressed as numerical values.

As described above, the illustrated agitation detection unit 12A judges the current agitation/non-agitation state of the target patient, in a machine learning scheme, on the basis of a temporal change of the biological information and a temporal change pattern as a predictor of the problem having occurred in the past. In other words, the agitation state discrimination unit 21A automatically, without relying on manpower, discriminates and judges whether or not the target patient is in the agitation state at the current moment and produces the current agitation score S(t) indicative of the current agitation/non-agitation state of the target patient. When the current agitation score S(t) is higher than a particular value (preliminarily set threshold value), that is, when the target patient is in the agitation state at the current moment, the biological information processing system 100A notifies the nursing/caregiving workers of an alarm as an agitation state notification signal. The agitation state notification signal is notified as a voice and/or an image.

Now, operation of the agitation state discrimination unit 21 will be described more specifically. The agitation state discrimination unit 21 generates the current agitation score S(t) by processing the detection-use feature time-series data Y(t) obtained by calculating, for example, the heartbeat value (or the reciprocal of the heartbeat value) which is supplied from the feature calculation unit 11A. Specifically, the agitation state discrimination unit 21 multiplies the discrimination parameter by the feature vector being the detection-use feature time-series data Y(t) to calculate the current agitation score S(t).

Herein, assuming that the discrimination parameter is linear and is represented by a coefficient vector w:

S(t)=1 (when wY(t)≥0)

S(t)=0 (when wY(t)<0).

Herein, the agitation state discrimination unit 21 may produce the current agitation score in the form of a binary signal of 0 or 1 as described above or as the degree of similarity (probability) represented by a numerical value in a range between 0 and 1, both inclusive.

Although the above-mentioned agitation state discrimination unit 21 is described as carrying out simple linear discrimination, a statistical linear technique such as SVM (Support Vector Machine) and LVQ (Learning Vector Quantization) may be used as the machine learning technique or a statistical nonlinear technique such as a neural network may be used as the machine learning technique. Those techniques are general techniques and, therefore, will not be described herein in detail.

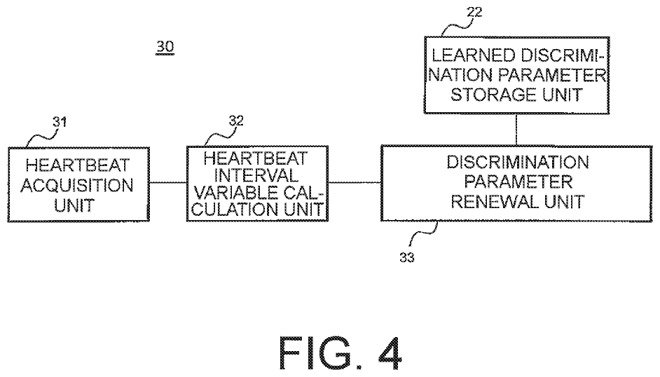

FIG. 4 is for illustrating a biological information processing system 30 for obtaining the discrimination parameter to be stored in the learned discrimination parameter storage unit 22 illustrated in FIG. 3. That is, the biological information processing system 30 carrying out operation of the learning phase in the machine learning may be called a learning system. It is assumed that the biological information processing system 30 illustrated in FIG. 4 receives, as an input signal, the heartbeat value h(t) and/or the reciprocal r(t) of the heartbeat value from a heartbeat sensor or the like and successively renews the discrimination parameter on the basis of the time-series data of the input signal and data indicative of a relationship with agitation/non-agitation. In this connection, the biological information processing system 30 includes a heartbeat acquisition unit 31, a heartbeat interval variable calculation unit 32, and a discrimination parameter renewal unit 33.

Specifically, the heartbeat acquisition unit 31 constructed of a sensor such as the heartbeat sensor supplies the heartbeat interval variable calculation unit 32 with the time-series data X(t) indicative of the current biological information from the target patient. The heartbeat interval variable calculation unit 32 carries out operation similar to that of the feature calculation unit 11A illustrated in FIG. 3 and supplies the detection-use time-series data Y(t) indicative of the feature as the feature vector to the discrimination parameter renewal unit 33.

The learned discrimination parameter storage unit 22 stores, as the learned discrimination parameter, the discrimination parameter indicative of a relationship between the past feature vectors Y′(t) of a lot of target patients and the past agitation/non-agitation of those target patients.

On the other hand, the discrimination parameter renewal unit 33 has a configuration which is similar to that of the agitation state discrimination unit 21 illustrated in FIG. 3. That is, the discrimination parameter renewal unit 33 obtains a new learned discrimination parameter by performing, in accordance with a predetermined algorithm, calculation on the learned discrimination parameter and the detection-use feature time-series data Y(t). The learned discrimination parameter is generated on the basis of the feature time-series data Y′(t) for learning processing, obtained by calculation from the past biological information time-series data X′(t) and the data indicative of the past agitation/non-agitation state. The detection-use feature time-series data Y(t) is obtained by calculation from the current biological information of the target patient. In this event, the discrimination parameter renewal unit 33 may carry out operation so as to minimize a difference between the current agitation score S(t) and the data indicative of the past agitation/non-agitation state. Specifically, the discrimination parameter renewal unit 33 renews a coefficient parameter w on the basis of the detection-use feature time-series data Y(t), the learned discrimination parameter, and the data indicative of the past agitation/non-agitation state and stores a renewed result in the learned discrimination parameter storage unit 22 as the new learned discrimination parameter. As described above, the learned discrimination parameter is renewed at any time with increment of the detection-use feature time-series data Y(t) given as the learning data and the data indicative of the past agitation/non-agitation state. As a result, a discrimination accuracy of the learned discrimination parameter is improved with the increment of accumulated data.

The present inventors confirmed, by actually applying the biological information processing system illustrated in FIGS. 1 to 4 to the patient, whether or not the occurrence of the problem behavior can be predicted by detecting, prior to the occurrence of the problem behavior of the patient, that the patient as the target patient is in the agitation state. Herein, observation was made by paying attention to a relationship between a behavior until the problem behavior (in this case, a bed-leaving behavior) occurs after the patient awaked and a change in heartbeat of the patient. As a result, an interval average value of the heartbeat and variance of the heartbeat rapidly become large thirty minutes before the bed-leaving behavior as the problem behavior. Thus, the present inventors found out by observing the heartbeat that the patient shifted from the non-agitation state to the agitation state which is a state before the problem behavior. As a result of the observation, the patient exhibited the bed-leaving behavior when about thirty minutes elapsed after transition from the non-agitation state to the agitation state.

The above-mentioned current agitation state of the patient can be accurately and appropriately detected by the biological information processing system according to the present invention. From this, the present inventors confirmed that the heartbeat serves as an indicator of transition from the agitation state to the non-agitation state and is effective for prediction of the occurrence of the problem behavior.

In FIGS. 1 to 3, description has been made about a case of calculating a plurality of features (feature vectors) on the basis of the time-series data of the heartbeat interval r_t (reciprocal of the heartbeat value h_t) at time instants t (t=1, . . . , T). However, the present invention is not limited to this calculation method. For example, a plurality of values calculated from the time-series data of the heartbeat value h_t may be used as the features indicative of the heartbeat values. In this event, the feature calculation unit calculates, using the heartbeat h_t within a past predetermined time interval, a difference h_t1−h_t2 (there are a plurality of patterns in intervals between time instants) of h_t values at different time instants. In addition, the feature calculation unit calculates a minimum value min (h_t) and a maximum value max (h_t) within the past predetermined time interval.

Subsequently, the agitation detection unit (agitation state discrimination unit) calculates the current agitation score using the detection-use time-series data Y(t) indicative of the features (a train of numerical values) and the discrimination parameter to discriminate two patterns of agitation/non-agitation.

As regards the above-mentioned r_t and h_t, normalization processing may be carried out before calculating the features. In this event, assuming that an average value of r_t within the past predetermined time interval is r_m and a standard deviation value within the predetermined time interval is r_s:

r_t′=(r_t−r_m)/r_s; (1)

Assuming that an average value of h_t within the past predetermined time interval is h_m and a standard deviation value within the predetermined time interval is h_s:

h_t′=(h_t−h_m)/h_s; (2)

From the above-mentioned equations (1) and (2), a normalized average value r_t′ and a normalized standard deviation value h_t′ are obtained.

As described above, by using the normalized features (feature vectors), it is possible not only to predict the occurrence of the problem behavior of the individual patient but also to give a general barometer for the problem behavior for a plurality of patients. Furthermore, by the normalization processing, it is possible to control daytime variation of the biological information even in a particular patient. In addition, for the normalization processing also, calculation may be carried out after filtering the detection-use feature time-series data Y(t) using a normalization filter, in the manner similar to other processing.

In FIGS. 1 to 4, it has been described that the occurrence of the problem behavior can be predicted by using the time-series variation amount of the heartbeat to detect transition from the non-agitation state to the agitation state. It is possible to similarly detect transition from the non-agitation state to the agitation state by using, as another technique, an electrocardiographic waveform of an electrocardiogram. For example, the electrocardiogram includes a plurality of waveforms such as the P wave, the Q wave, the R wave, the S wave, and the T wave, as described above. Among various types of waveforms included in the electrocardiogram, characteristic variation amounts other than the RR interval indicative of the heartbeat, for example, a PQ time interval, a QRS width, a QT time interval, or the like may be observed and recorded in a time-series fashion to subject a relationship between those variation amounts and the past agitation/non-agitation of the patient to the machine learning. By this technique also, the agitation detection units 12 and 12A can calculate the current agitation score indicative of the current agitation/non-agitation state to produce the agitation state notification signal. In a case of using the PQ time interval in the electrocardiogram, it is possible to detect the transition from the non-agitation state of the patient to the agitation state prior to the occurrence of the problem behavior, for example, by collecting a plurality of PQ time intervals to carry out a convolution operation of the collected PQ time intervals by the agitation detection units 12 and 12A.

In this case also, a correlation relationship between the above-mentioned characteristic variation amounts and the past agitation/non-agitation can be preliminarily prepared as the discrimination parameter by the machine learning technique. The agitation detection units 12 and 124 can calculate the current agitation score on the basis of the input detection-use feature time-series data and the discrimination parameter to produce the calculated current agitation score as transition information from the non-agitation state of the patient to the agitation state. As described above, in this example embodiment, it is possible to detect, prior to occurrence of the problem behavior of the patient, that the patient is in the agitation state and to notify the nursing/caregiving workers prior to occurrence of the problem behavior.

Furthermore, in the above-mentioned example embodiments, description has mainly been made as regards an example of obtaining the current agitation score indicative of the current agitation/non-agitation state using the information relating to the heartbeat as the biological information. However, the present invention is similarly applicable to a case of using biological information other than the heartbeat, for example, biological information such as breathing, blood pressure, deep-body temperature, a level of consciousness, skin temperature, skin conductance response, a skin potential, a myoelectric potential, an electrocardiographic waveform, an electroencephalographic waveform, a sweating amount, a blood oxygen saturation level, a pulse waveform, optical brain function mapping, a urine volume, and pupil reflex.

Furthermore, as the technique of generating the learned discrimination parameter, a statistical linear discrimination technique such as SVM and LVQ or a statistical nonlinear discrimination technique such as a neural network may be used.

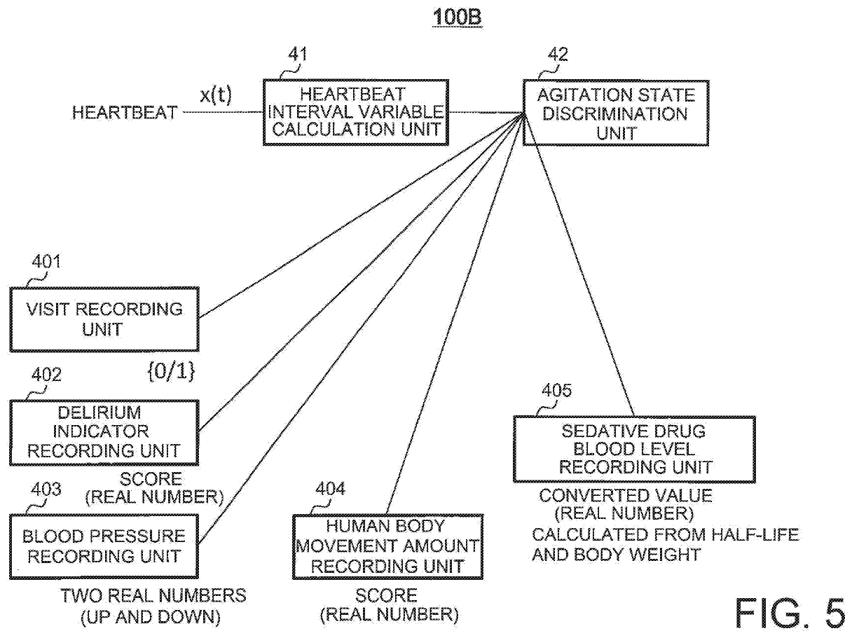

Referring to FIG. 5, description will proceed to a biological information processing system 100B according to a second example embodiment the present invention. According to observation of the present inventors, it was confirmed that the problem behavior of the patient is generated by various factors. For example, when the patient is visited by a person who has lived with the patient, such as a family, within 24 hours, he/she often wishes to return home. The patient may exhibit the problem behavior due to the visit. Thus, the problem behavior of the patient occurs not only due to information described in an electronic medical chart.

As described above, it may also be possible to predict the occurrence of the problem behavior of the patient by using information which is not described in the electronic medical chart, for example, information indicative of presence or absence of the visit.

Furthermore, it may also be possible to predict the occurrence of the problem behavior of the patient by combining the following additional information (A1 to A7) with the above-mentioned information (information relating to the heartbeat, the information described in the electronic medical chart, and the information indicative of presence or absence of the visit).

A1. Check items in a periodical problem behavior assessment sheet in a nursing record (a part of the electronic medical chart);

A2. An assessment indicator of “delirium”;

A3. Information indicating who are other inpatients in the same room and the nursing/caregiving workers assigned to the patient;

A4. Information indicative of a kind of a sedative hypnotic drug, of an elapsed time from administration, of a half-life of the drug, of a body weight, and of an age;

A5. A variation pattern of biological information (e.g. a body movement amount, skin body temperature, a sweating amount, blood pressure, a myoelectric potential, a breathing rate) other than the heartbeat, that is detected by biosensors;

A6. A posture of a human body and an amount of movement which are detected from a camera image, for example, the number of times of touching a face with a hand in the movement of the human body that is detected from the camera image, the number of times of touching an arm with a hand in the movement of the human body that is detected from the camera image; and

A7. Utterance of the target patient which is detected by a microphone, for example, shout or self-talk of the target patient.

The biological information processing system 100B illustrated in FIG. 5 predicts the occurrence of the problem behavior by combining the above-mentioned additional information according to A1 to A7 mentioned above. The illustrated biological information processing system 100B comprises a heartbeat interval variable calculation unit 41 and an agitation state discrimination unit 42. The heartbeat interval variable calculation unit 41 carries out processing which is similar to the heartbeat interval variable calculation unit 32 illustrated in FIG. 4, receives the current time-series data X(t) relating to the heartbeat from the target patient, and supplies the agitation state discrimination unit 42 with the detection-use time-series data Y(t) indicative of the feature. In this event, the heartbeat interval variable calculation unit 41 may supply the agitation state discrimination unit 42 with the variation amounts of the average values m1 and m2 of the minimum value min(r_t) and the maximum value max(r_t) in the different time intervals and the variation amounts s1 and s2 of variance within intervals 1 and 2, as the time-series data Y(t) indicative of the detection-use feature.

Furthermore, the illustrated agitation state discrimination unit 42 is also supplied with the additional information according to A1 to A7 mentioned above as the time-series data Y(t) indicative of the detection-use feature. That is, the agitation state discrimination unit 42 is supplied with not only the calculated result from the heartbeat interval variable calculation unit 41 but also the additional information which is selected from A1 to A7 mentioned above.

Specifically, the biological information processing system 100B includes a visit recording unit 401 indicative of presence or absence of a visit record with a cohabiter or the like, a delirium indicator recording unit 402, a blood pressure recording unit 403, a human body movement amount recording unit 404, and a sedative drug blood level recording unit 405. The agitation state discrimination unit 42 is supplied with data from the respective units as the time-series data Y(t) indicative of the detection-use feature.

In the visit recording unit 401, binary data indicative of presence or absence of the visit to the patient and a visit time interval are recorded as reception record information. In addition, in the delirium indicator recording unit 402, a delirium indicator of the patient is recorded. In the blood pressure recording unit 403, diastolic blood pressure and systolic blood pressure, and a measurement time interval are recorded. The delirium indicator may be a delirium score which is described in the above-mentioned Patent Literature 4.

Furthermore, in the human body movement amount recording unit 404, a movement amount of the patient obtained from an image of a camera or the like is recorded. In the sedative drug blood level recording unit 405, a half-life of the drug that is calculated from a body weight of the patient is recorded.

Herein, information from the visit recording unit 401, the delirium indicator recording unit 402, the blood pressure recording unit 403, the human body movement amount recording unit 404, and the sedative drug blood level recording unit 405 will be called additional information hereinafter. The additional information is supplied to the agitation state discrimination unit 42 selectively or in combination. Thus, the additional information supplied to the agitation state discrimination unit 42 need not include all of the reception record information of the patient, administration record information of the drug, body weight information, and age information, but may include at least one of them. Furthermore, the additional information may include additional information according to A1 to A7 mentioned above, other than the information recorded in the above-mentioned recording units 401 to 405 (roommate inpatient information, the body temperature, the utterance, or the like).

The agitation state discrimination unit 42 calculates the current agitation score on the basis of not only the calculated result from the heartbeat interval variable calculation unit 41 but also the additional information from the above-mentioned recording units 401 to 405 to produce the agitation state notification signal indicative of the current agitation/non-agitation state. In this event, the agitation state discrimination unit 42 receives, as the feature vectors, not only the detection-use time-series data Y(t) relating to the heartbeat but also the detection-use time-series data related to the additional information, and calculates the current agitation score on the basis of these feature vectors and the discrimination parameter which is preliminarily learned on the basis of data relating to the past agitation/non-agitation.

In this event, the detection-use time-series data from the heartbeat interval variable calculation unit 41 may not always be used and, for example, the current agitation/non-agitation state of the target patient may be determined only by the additional information. In this event, the agitation state discrimination unit 42 receives the additional information as the feature vector and calculates the current agitation score in accordance with the feature vector relating to the additional information and a model (discrimination parameter) which is preliminarily prepared on the basis of data relating to the past agitation/non-agitation.

The biological information processing system 100B illustrated in FIG. 5 can obtain an agitation score which is specialized for the target patient him/herself. Furthermore, it is also possible to compare the agitation score of the target patient with agitation scores of other patients and to rank the patients in the order of likelihood of occurrence of the problem behavior. Thus, the nursing/caregiving workers can cope with the patients in the order of likelihood of occurrence of the problem behavior and a burden on the nursing/caregiving workers or the like can considerably be reduced.

In a case where it is notified by the agitation score from the agitation state discrimination unit 42 that the target patient is in the agitation state, the nursing/caregiving workers can visually confirm a condition of the target patient if an image of the target patient is displayed on a monitor of a mobile terminal held by the nursing/caregiving workers or the like or on a monitor in a nurse station. Thus, even in a case where the agitation state notification signal is erroneously produced from the agitation state discrimination unit 42, the nursing/caregiving workers can deal with the target patient after visual confirmation. It is therefore possible to further reduce a work burden of the nursing/caregiving workers.

As sensors for sensing the biological information of the patients, contact or contactless sensors may be used. For instance, the sensors for detecting the heartbeat (pulsation) may be a wristwatch-type sensor, a chest-patch type sensor, or a sensor for carrying out non-contact detection of the heartbeat by the image of the camera or the like.

In FIG. 4, description has mainly been made about a case of generating the learned discrimination parameter using the information relating to the heartbeat as the biological information of the target patient. However, the present invention is not limited thereto, and it is possible to similarly obtain the learned discrimination parameter by using the biological information other than the heartbeat.

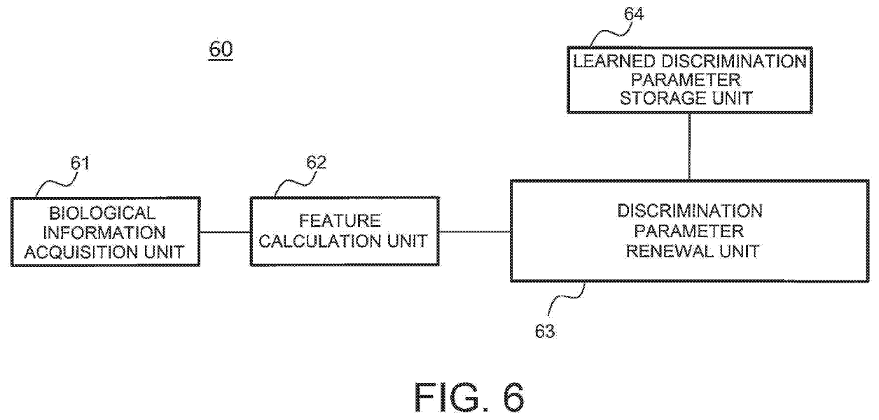

Referring to FIG. 6, a biological information processing system (learning system) 60 is illustrated which obtains the discrimination parameter by machine-learning the biological information other than the heartbeat. The illustrated biological information processing system 60 comprises a biological information acquisition unit 61, a feature calculation unit 62, a discrimination parameter renewal unit 63, and a learned discrimination parameter storage unit 64. Herein, a case of operating in a learning stage will be described.

The biological information acquisition unit 61 comprises a sensor for sensing, in a lot of target patients, at least one of breathing, blood pressure, deep-body temperature, a level of consciousness, skin body temperature, a skin conductance response, a skin potential, a myoelectric potential, an electrocardiographic waveform, an electroencephalographic waveform, a sweating amount, a blood oxygen saturation level, a pulse waveform, optical brain function mapping, a urine amount, pupil reflex, and so on. Current time-series data from the biological information acquisition unit 61 is supplied to the feature calculation unit 62. The feature calculation unit 62 generates time-series data indicative of detection-use feature according to the input biological information. The discrimination parameter renewal unit 63 generates the learned discrimination parameter on the basis of a relationship between the time-series data indicative of the detection-use feature and data indicative of a relationship with the past agitation/non-agitation and stores the parameter in the leaned discrimination parameter storage unit 64.

As described above, the biological information processing system 60 in which the learned discrimination parameter is stored in the learned discrimination parameter storage unit 64 may also be used as a biological information processing system for producing the agitation score as the agitation state notification signal, like in FIG. 1.

In this event, the discrimination parameter renewal unit 63 illustrated in FIG. 6 is similar to the discrimination parameter renewal unit 33 illustrated in FIG. 4. In a case of this example, the biological information acquisition unit 61 acquires the current time-series data of the biological information as a target on the basis of the current biological information of the target patient, and the feature calculation unit 62 generates the time-series data indicative of the detection-use feature. Thereafter, the time-series data indicative of the detection-use feature is multiplied by the discrimination parameter read out of the learned discrimination parameter storage unit 64 and the current agitation score is calculated.

[Hardware Configuration of the Biological Information Processing System]

The biological information processing system 100A and the biological information processing system 100B mentioned above may be implemented by hardware or may be implemented by software. In addition, the biological information processing system 100A and the biological information processing system 100B may be implemented by a combination of hardware and software.

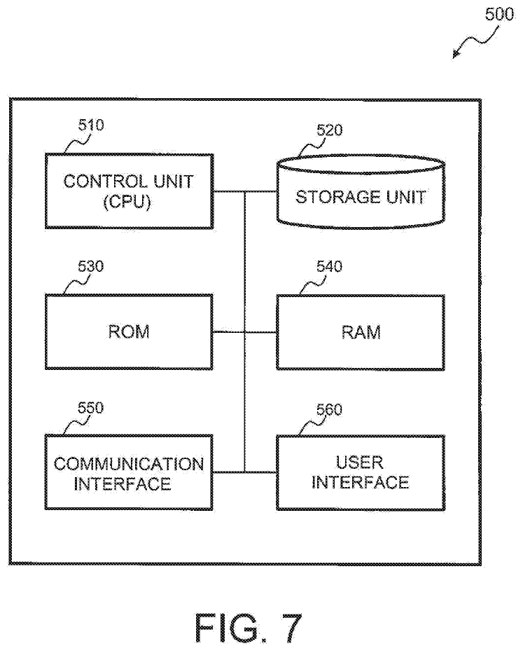

FIG. 7 is a block diagram for illustrating one example of an information processing apparatus (computer) constituting the biological information processing system 100A and the biological information processing system 100B.

As shown in FIG. 7, the information processing apparatus 500 comprises a control unit (CPU: Central Processing Unit) 510, a storage unit 520, an ROM (Read Only Memory) 530, an RAM (Random Access Memory) 540, a communication interface 550, and a user interface 560.

The control unit (CPU) 510 may implement various functions of the biological information processing system 100A and the biological information processing system 100B by developing, in the RAM 540, a program which is stored in the storage unit 520 or the ROM 530, and by executing the program. In addition, the control unit (CPU) 510 may comprise an internal buffer which is adapted to temporarily store data or the like.

The storage unit 520 comprises a bulk storage medium which can hold various types of data and may be implemented by a storage medium such as an HDD (Hard Disk Drive) and an SSD (Solid State Drive). The storage unit 520 may be a cloud storage existing in a communication network when the information processing apparatus 500 is connected to the communication network via the communication interface 550. The storage unit 520 may hold the program readable by the control unit (CPU) 510.

The ROM 530 is a nonvolatile storage device which may comprise a flash memory having a small capacity as compared to the storage unit 520. The ROM 530 may hold a program which is readable by the control unit (CPU) 510. The program readable by the control unit (CPU) 510 may be held in at least one of the storage unit 520 and the ROM 530.

The program readable by the control unit (CPU) 510 may be supplied to the information processing apparatus 400 in a state where it is non-transitorily stored in various storage media readable by the computer. Such storage media include, for example, a magnetic tape, a magnetic disk, a magneto-optical disc, a CD-ROM (Compact Disc-Read Only Memory), a CD-R (Compact Disc-Readable), a CD-RW (Compact Disc-ReWritable), and a semiconductor memory.

The RAM 440 comprises a semiconductor memory such as a DRAM (Dynamic Random Access Memory) and an SRAM (Static Random Access Memory) and may be used as an internal buffer which temporarily stores data and so on.

The communication interface 550 is an interface which connects the information processing system 500 and the communication network via wire or wirelessly.

The user interface 560 comprises, for example, a display unit such as a display and an input unit such as a keyboard, a mouse, and a touch panel.

While the present invention has been described with reference to the example embodiments thereof, the present invention is not limited to the foregoing embodiments. It will be understood by those skilled in the art that various changes in form and details of the present invention may be made without departing from the spirit and scope of the present invention.

A part or a whole of the example embodiments described above may also be described as the following supplementary notes without being limited thereto.

(Supplementary Note 1)

A biological information processing system comprising a feature calculation unit configured to calculate, from input biological information of a target patient, detection-use feature time-series data indicative of a feature related to the target patient; and an agitation detection unit configured to process the detection-use feature time-series data on the basis of a discrimination parameter which is preliminarily acquired, to calculate a current agitation score of the target patient, and to detect a current agitation state of the target patient prior to a problem behavior of the target patient.

(Supplementary Note 2)

The biological information processing system according to Supplementary Note 1, comprising a storage unit configured to store the discrimination parameter, wherein the storage unit is configured to store the discrimination parameter which is calculated on the basis of a first feature time-series data for learning processing, obtained from biological information in an agitation state and a second feature time-series data for learning processing, obtained from biological information in a non-agitation state.

(Supplementary Note 3)

The biological information processing system according to Supplementary Note 1 or 2, wherein the agitation detection unit is configured to calculate the current agitation score of the target patient using the discrimination parameter and the detection-use feature time-series data from the feature calculation unit.

(Supplementary Note 4)

The biological information processing system according to Supplementary Note 3, wherein the agitation detection unit is configured to calculate the current agitation score of the target patient by multiplying the discrimination parameter by the detection-use feature time-series data from the feature calculation unit.

(Supplementary Note 5)

The biological information processing system according to any one of Supplementary Notes 1 to 4, wherein the discrimination parameter comprises a linear parameter or a non-linear parameter which is obtained by a machine learning technique.

(Supplementary Note 6)

The biological information processing system according to any one of Supplementary Notes 1 to 5, wherein the biological information comprises information selected from the group consisting of a heartbeat, breathing, blood pressure, body temperature, a level of consciousness, skin temperature, skin conductance response, an electrocardiographic waveform, and an electroencephalographic waveform.

(Supplementary Note 7)

The biological information processing system according to any one of Supplementary Notes 1 to 6, wherein the agitation detection unit is configured to detect the current agitation state of the target patient using additional information related to the target patient in addition to the detection-use feature time-series data.

(Supplementary Note 8)

A biological information processing system, comprising a feature calculation unit configured to calculate, from input biological information of a target patient, detection-use feature time-series data indicative of a feature related to the target patient; a discrimination parameter storage unit configured to store a discrimination parameter which is preliminarily acquired; and a discrimination parameter renewal unit configured to process the detection-use feature time-series data on the basis of the discrimination parameter to renew the discrimination parameter.

(Supplementary Note 9)

A biological information processing method, comprising calculating, from input biological information of a target patient, detection-use feature time-series data indicative of a feature related to the target patient; and processing the detection-use feature time-series data on the basis of a discrimination parameter which is preliminarily acquired, calculating a current agitation score of the target patient, and detecting a current agitation state of the target patient prior to a problem behavior of the target patient.

(Supplementary Note 10)

The biological information processing method according to Supplementary Note 9, comprising calculating the discrimination parameter on the basis of a first feature time-series data for teaming processing, obtained from biological information in an agitation state and a second feature time-series data for learning processing, obtained from biological information in a non-agitation state.

(Supplementary Note 11)

A biological information processing method, comprising calculating, from input biological information of a target patient, detection-use feature time-series data indicative of a feature related to the target patient; storing, in a discrimination parameter storage unit, a discrimination parameter which is preliminarily acquired; and processing the detection-use feature time-series data on the basis of the discrimination parameter, and renewing the discrimination parameter to make the discrimination parameter be stored in the discrimination parameter storage unit.

(Supplementary Note 12)

A recording medium for storing a computer program which causes a computer to execute the steps of calculating, from input biological information of a target patient, detection-use feature time-series data indicative of a feature related to the target patient; and processing the detection-use feature time-series data on the basis of a discrimination parameter which is preliminarily acquired, calculating a current agitation score of the target patient, and detecting a current agitation state of the target patient prior to a problem behavior of the target patient.

(Supplementary Note 13)

A recording medium for storing a computer program which causes a computer to execute the steps of calculating, from input biological information of a target patient, detection-use feature time-series data indicative of a feature related to the target patient; storing, in a discrimination parameter storage unit, a discrimination parameter which is preliminarily acquired; and processing the detection-use feature time-series data on the basis of the discrimination parameter, and renewing the discrimination parameter to make the discrimination parameter be stored in the discrimination parameter storage unit.

### INDUSTRIAL APPLICABILITY

The biological information system according to the present invention can greatly reduce a burden and a workload on the nursing/caregiving workers or the like caring for the patients by using the system in an acute-care hospital, a rehabilitation hospital, a care facility, and so on.

This application is based upon and claims the benefit of priority from Japanese patent application No. 2017-165605, filed on Aug. 30, 2017, the disclosure of which is incorporated herein in its entirety by reference.

### REFERENCE SIGNS LIST

100, 100A, 100B biological information processing system

11, 11A feature calculation unit

12, 12A agitation detection unit

21 agitation state discrimination unit

22 learned discrimination parameter storage unit

30 biological information processing system (learning system

31 heartbeat acquisition unit

32 heartbeat interval variable calculation unit

33 discrimination parameter renewal unit

41 heartbeat interval variable calculation unit

42 agitation state discrimination unit

401 visit recording unit

402 delirium indicator recording unit

403 blood pressure recording unit

404 human body movement amount recording unit

405 sedative drug blood level recording unit

60 biological information processing system (learning system

61 biological information acquisition unit

62 feature calculation unit

63 discrimination parameter renewal unit

64 learned discrimination parameter storage unit

500 information processing apparatus

510 control unit(CPU)

520 storage unit

530 ROM

540 RAM

550 communication interface

560 user interface

1. A biological information processing system comprising:
a feature calculation unit configured to calculate, from input biological information of a target patient, detection-use feature time-series data indicative of a feature related to the target patient; and
an agitation detection unit configured to process the detection-use feature time-series data on the basis of a discrimination parameter which is preliminarily acquired, to calculate a current agitation score of the target patient, and to detect a current agitation state of the target patient prior to a problem behavior of the target patient.

2. The biological information processing system as claimed in claim 1, comprising a storage unit configured to store the discrimination parameter,
wherein the storage unit is configured to store the discrimination parameter which is calculated on the basis of a first feature time-series data for learning processing, obtained from biological information in an agitation state and a second feature time-series data for learning processing, obtained from biological information in a non-agitation state.

3. The biological information processing system as claimed in claim 1, wherein the agitation detection unit is configured to calculate the current agitation score of the target patient using the discrimination parameter and the detection-use feature time-series data from the feature calculation unit.

4. The biological information processing system as claimed in claim 3, wherein the agitation detection unit is configured to calculate the current agitation score of the target patient by computation processing including an operation of multiplying the discrimination parameter by the detection-use feature time-series data from the feature calculation unit.

5. The biological information processing system as claimed in claim 1, wherein the discrimination parameter comprises a linear parameter which is obtained by a linear machine learning technique or a non-linear parameter which is obtained by a non-linear machine learning technique.

6. The biological information processing system as claimed in claim 1, wherein the biological information comprises information selected from the group consisting of a heartbeat, breathing, blood pressure, body temperature, a level of consciousness, skin temperature, skin conductance response, an electrocardiographic waveform, and an electroencephalographic waveform.

7. The biological information processing system as claimed in claim 1, wherein the agitation detection unit is configured to detect the current agitation state of the target patient using additional information related to the target patient in addition to the detection-use feature time-series data.

8. (canceled)

9. A biological information processing method comprising:
calculating, from input biological information of a target patient, detection-use feature time-series data indicative of a feature related to the target patient; and
processing the detection-use feature time-series data on the basis of a discrimination parameter which is preliminarily acquired, calculating a current agitation score of the target patient, and detecting a current agitation state of the target patient prior to a problem behavior of the target patient.

10. The biological information processing method as claimed in claim 9, comprising calculating the discrimination parameter on the basis of a first feature time-series data for learning processing, obtained from biological information in an agitation state and a second feature time-series data for learning processing, obtained from biological information in a non-agitation state.

11. (canceled)

12. A non-transitory computer readable recording medium recording a computer program which causes a computer to execute the steps of:
calculating, from input biological information of a target patient, detection-use feature time-series data indicative of a feature related to the target patient; and
processing the detection-use feature time-series data on the basis of a discrimination parameter which is preliminarily acquired, calculating a current agitation score of the target patient, and detecting a current agitation state of the target patient prior to a problem behavior of the target patient.

13. (canceled)

14. The biological information processing method as claimed in claim 9, wherein the calculating the current agitation score of the target patient calculates the current agitation score of the target patient using the discrimination parameter and the detection-use feature time-series data.

15. The biological information processing method as claimed in claim 14, wherein the calculating the current agitation score of the target patient calculates the current agitation score of the target patient by computation processing including an operation for multiplying the discrimination parameter by the detection-use feature time-series data.

16. The biological information processing method as claimed in claim 9, wherein the discrimination parameter comprises a linear parameter which is obtained by a linear machine learning technique or a non-linear parameter which is obtained by a non-linear machine learning technique.

17. The biological information processing method as claimed in claim 9, wherein the biological information comprises information selected from the group consisting of a heartbeat, breathing, blood pressure, body temperature, a level of consciousness, skin temperature, skin conductance response, an electrocardiographic waveform, and an electroencephalographic waveform.

18. The biological information processing method as claimed in claim 9, wherein the detecting the current agitation state of the target patient detects the current agitation state of the target patient using additional information related to the target patient in addition to the detection-use feature time-series data.

19. The non-transitory computer readable recording medium as claimed in claim 12, wherein the computer program causes the computer to execute the step of calculating the discrimination parameter on the basis of a first feature time-series data for learning processing, obtained from biological information in an agitation state and a second feature time-series data for learning processing, obtained from biological information in a non-agitation state.

20. The non-transitory computer readable recording medium as claimed in claim 12, wherein the computer program causes the computer to execute the step of calculating the current agitation score of the target patient using the discrimination parameter and the detection-use feature time-series data.

21. The non-transitory computer readable recording medium as claimed in claim 20, wherein the computer program causes the computer to execute the step of calculating the current agitation score of the target patient by computation processing including an operation for multiplying the discrimination parameter by the detection-use feature time-series data.

22. The non-transitory computer readable recording medium as claimed in claim 12, wherein the discrimination parameter comprises a linear parameter which is obtained by a linear machine learning technique or a non-linear parameter which is obtained by a non-linear machine learning technique.

23. The non-transitory computer readable recording medium as claimed in claim 12, wherein the biological information comprises information selected from the group consisting of a heartbeat, breathing, blood pressure, body temperature, a level of consciousness, skin temperature, skin conductance response, an electrocardiographic waveform, and an electroencephalographic waveform.
